(19) United States
(12) Patent Application Publication
(10) Pub. No.: US 2004/0115835 A1
(43) Pub. Date: Jun. 17, 2004

(54) Methods for predicting impaired performance in equines

(76) Inventors: Robert Llewellyn Clancy, New South Wales (AU); Gerald Pang, New South Wales (AU)

Correspondence Address: FISH & RICHARDSON PC, 225 FRANKLIN ST, BOSTON, MA, 02110, US

(21) Appl. No.: 10/363,297

(22) PCT Filed: Aug. 29, 2001
(86) PCT No.: PCT/AU01/01085

(30) Foreign Application Priority Data
Aug. 29, 2000 (AU) PQ 9756
Oct. 25, 2000 (AU) PQ 1015

(51) Int. Cl. G01N 33/53; G01N 33/543

(52) U.S. Cl. 436/518

(57) ABSTRACT

The present invention is concerned with methods of assessing performance and performance potential in horses using measurement of immunoglobulin levels, particularly levels of IgA and/or IgG. The invention is also concerned with methods for predicting impaired performance, fatigue and/or susceptibility to infection in horses exposed to stressors, in particular physical stressors.

### TECHNICAL FIELD

The present invention is concerned with measurement of immunoglobulins in biological fluids as a method of detecting impaired performance, fatigue and/or infection. More specifically the present invention is concerned with measurement of salivary IgA and/or IgG in horses as a determinant of fatigue, impaired performance or risk of impaired performance, poor potential to perform and/or infection.

### BACKGROUND ART

A significant database exists in man which links salivary IgA levels with impaired performance in athletes in a reciprocal fashion. Recent data has shown that essentially all elite athletes have EB virus DNA incorporated in B cell genome and that in those with low IgA levels and inappropriate high volume of training activates EBV excretion as a mechanism of fatigue and impaired performance. There has been no studies in any other animal model to develop an assay to monitor impaired performance, fatigue and/or infection. Whether or not immunoglobulin levels in various biological fluids can be used to monitor or assess impaired performance in any other species (EBV is specific for a limited number of animal species) is not known. More specifically, EBV does not infect horses.

Racing horses and those participating in other sports such as polo and the like are exposed to severe competition stress as well as to training/exercise stress. It would be important to monitor exercise levels and stresses imposed by competition, to enable appropriate training and rest programs to be implemented where required at the appropriate time.

Thus there is a need for a suitable test to monitor impaired performance, fatigue and/or predict susceptibility to infection, or for assessing risk of poor performance or potential to perform, in horses exposed to physical or other stress, to enable changes in training programs to be made or to use intervention therapy where appropriate.

It is an object of the present invention to overcome or ameliorate the deficiencies of the prior art, or to provide a useful alternative.

### SUMMARY OF THE INVENTION

Measurement of IgA in humans has proved to be a valuable tool in the assessment of stress-induced fatigue and/or infection susceptibility. It was unexpectedly found that salivary immunoglobulin levels, in particular IgA and/or IgG, in horses exposed to physical stress such as training or competition can be an indicator of fatigue and/or susceptibility to infection or poor potential. Further, if the decrease in IgA and/or IgG levels which occurs after exposure to a stressor is not recovered rapidly, prolonged reduction in levels of these immunoglobulins results in susceptibility to infection and/or fatigue and thus reduced performance. As mentioned above, reduced IgA and/or IgG levels are an indicator not only of fatigue but also of susceptibility to infection. Thus the rate of recovery of IgA and/or IgG levels is also a good indicator of susceptibility to infection or poor potential to perform.

According to a first aspect, the invention provides a method for predicting performance potential, impaired performance, fatigue and/or potential susceptibility to infection in horses exposed to stressor(s) including:

(a) determination of immunoglobulin A (IgA) and/or immunoglobulin G (IgG) level in a training phase; and

(b) prediction of impaired performance, fatigue and/or susceptibility to infection by comparison of the training phase IgA and/or IgG level with a predetermined threshold value.

According to a second aspect, the invention provides a method for assessing and/or monitoring suitability of a physical training program for horses, including:

(a) determination of IgA and/or IgG level in a pre-training phase;

(b) determination of IgA and/or IgG level at intervals during a training phase and

(c) comparing IgA level during training phase with IgA and/or IgG levels in pre-training phase and thereby assessing suitability of the physical training program.

According to a third aspect, the invention provides a method for assessing the impact of stressor(s) on a horse's performance and/or fatigue levels, as defined herein, including:

(a) determination of salivary IgA and/or IgG level; and

(b) assessing the impact of the stressor or stressors on the horse's performance and/or fatigue levels by comparison of said IgA and/or IgG level with a predetermined threshold value.

According to a fourth aspect, the invention provides a method for assessing potential susceptibility to respiratory infection in a horse, including:

(a) determining the salivary IgA and/or IgG level in early training phase; and

(b) predicting the potential susceptibility to respiratory infection by comparison of the early training phase IgA and/or IgG level with a predetermined threshold value.

According to a fifth aspect, the invention provides a method for assessing potential susceptibility to fatigue/infection, impaired performance or potential to perform in horses, including:

(a) determining the IgA and/or IgG level after exposure to stressor(s);

(b) allowing a recovery period as herein defined;

(c) determining the IgA and/or IgG level after the recovery period;

(d) predicting the potential susceptibility to fatigue/infection or impaired performance by comparison of the IgA and/or IgG level at step (a) above with the IgA and/or IgG level at step (c) above.

According to a sixth aspect, the invention provides a method for designing a training program for a horse, including:

(a) determining the salivary IgA and/or IgG level before training begins,

(b) determining the salivary IgA and/or IgG level in early training phase,

(c) determining the salivary IgA and/or IgG level during optimal training phase and/or during competition or severe physical stress,

(d) establish an IgA and/or IgG profile for the horse using levels obtained in steps (a) to (c) to design a training program which maintains stable levels of IgA and/or IgG.

According to a seventh aspect, the invention provides a method for designing a training program for a horse, including the determination of salivary IgA and/or IgG levels at the time periods selected from one or more of the following:

(a) before training begins,

(b) in early training phase,

(c) during optimal training phase and/or during competition or severe physical stress.

According to an eighth aspect, the invention provides a method for assessing performance potential of a horse including:

(a) determination of immunoglobulin A (IgA) and/or immunoglobulin G (IgG) level before training begins; and

(b) prediction of poor performance potential by comparison of the IgA and/or IgG level with a predetermined threshold value.

According to a ninth aspect, the invention provides a method for assessing performance potential of a horse, including:

(a) determining the IgA and/or IgG level before training begins,

(b) determining the IgA and/or IgG level in early training phase,

(c) establishing an IgA and/or IgG profile for the horse using levels obtained in steps (a) to (b) and using said profile to predict poor performance potential by comparison of the IgA and/or IgG level with a predetermined threshold value.

According to a tenth aspect, the invention provides a method for predicting the capacity of a horse to complete a training program, including:

(a) determining the IgA and/or IgG level before training begins,

(b) determining the IgA and/or IgG level in early training phase,

(c) establishing an IgA and/or IgG profile for the horse using levels obtained in steps (a) to (b) and using said profile to predict the capacity of a horse to complete a training program by comparison of the IgA and/or IgG level with a predetermined threshold value.

The methods of the present invention are preferably used in conjunction with trainer assessment.

Preferably, the immunoglobulin level is determined in saliva because it is least invasive, but other biological fluids may also be used, for example blood, tears, reproductive and respiratory tract fluids, and the like. It will also be understood that levels IgA and/or IgG subclasses may be useful in the assessment of fatigue and/or infection. Immunoglobulin subclasses may also be used.

Preferably, the threshold value is an internal individual threshold value, usually obtained at rest or before training, if a horse is to be trained or exposed to physical stress. This enables an individual profile to be established and used in the assessment of potential performance capability or in designing an appropriate training program. However, in certain applications and in certain instances the normal threshold value may be determined in samples obtained from normal horses or from published data on normal horse IgA and/or IgG levels.

Preferably, the salivary immunoglobulin is from a sample of whole unstimulated saliva and most preferably the saliva sample is not a fasting sample.

Preferably, the horse is a racing or other competition horse.

Preferably, the stressor is known to influence the efficacy of the immune system.

Preferably, the horse is exposed to a physical and/or physiological stressor. Preferably, the physical and/or physiological stressor is long-term physical training, overtraining or competition.

Preferably, the immunoglobulin level is determined by ELISA or by a similar immunoassay. Preferably, the saliva is analysed for the immunoglobulin content in situ by contacting an assay device or system with the saliva of a horse.

In the context of the present invention, the word “stressor” includes within its meaning but is not limited to physical, physiological, psychological and nutritional stressors which include fatigue.

In the context of the present invention, the word “performance” includes within its meaning performance in relation to other horses, or performance in relation to individual performance level or potential to perform. Similarly, the term “fatigue” includes within its meaning fatigue in relation to other horses or fatigue in relation to individual fatigue level.

While the present invention has been exemplified by examples relating to horses exposed to a physical stressor, it will be clear to those skilled in the art that similar assessment of fatigue and/or susceptibility to infection will apply to other stressful activities.

In the context of the present invention the term “recovery period” includes a period of rest after exposure to a stressor or stressors. The appropriate length of time for recovery will be determined by the skilled addressee and will vary with the type of stressor and the type of horse.

Although the present invention has been described predominantly in the context of assessing impaired performance and fatigue it will be understood by those skilled in the art that the same principles can be applied to susceptibility to infection, including reactivation of latent infection and performance potential.

### BRIEF DESCRIPTION OF THE FIGURES

FIG. 1: Relationship between salivary IgA levels and performance in horses

FIGS. 2(A,B,C): IgA levels and mean visual analogue scores (VAS) (above bars) in horses failing to complete eight weeks training (ie horses spelled due to poor performance and/or injury).

FIGS. 3(A,B,C): IgA levels and mean VAS (above bars) in horses completing the eight week training programme.

Salivary IgA levels (bar graph) and trainers' performance indicators (shown top of the bar graph) were determined in individual horses. IgA levels were determined by ELISA and the results expressed as ELISA UNITS (EU) per mL. The performance score was as follows: 1-1.5, poor, 2-2.5, fair; 3-3.5; good, 4-4.5, very good; 5, outstanding. Each horse was identified by a code shown in the figure panel.

### DESCRIPTION OF THE PREFERRED EMBODIMENT

To enable measurement of equine IgA, an ELISA assay has been developed to detect the amount of IgA in horse saliva. The use of such an assay enables determination of salivary IgA levels in horses and thus provides means of assessing the consequences of training and the onset of fatigue and impaired performance. Fatigue may also be a consequence of infection resulting from physical stress and thus the assay may be used to assess susceptibility or predisposition to infection commonly encountered in horses, eg. common respiratory tract infections associated with equine flu, Streptococcus equi and equine herpes virus.

To exemplify the present invention, the assay has been used to measure saliva obtained from rested and exercised/trained horses. Saliva IgA levels were measured in 29 polo horses in the middle of a major competition where the horses were variably stressed. There was a remarkable variation in IgA levels in these healthy horses (1.69 to 579 ELISA units (EU)). This wide range is outside that expected for a homogenous population. It is therefore likely that it reflects a stress response, with low levels of IgA indicating risk of impaired performance, fatigue and/or susceptibility to infection.

Studies were also performed on racehorses in a similar manner, to obtain data which can be used to assess performance and design suitable training programs.

The invention will now be described more particularly by reference to specific but non-limiting examples.

### EXAMPLES

### Example 1

Saliva Collection

1. Horse saliva was collected by aspiration from the floor of the oral cavity using a cannula attached to a 10 mL syringe. The cannula was then detached from the syringe and placed on dry ice until processed in the laboratory.

2. The entire content from the cannula was emptied into a 1.5 mL Eppendorf tube and then centrifuged at 5000 rpm for 20 secs.

3. 0.2-0.5 mL of saliva fluid was collected by pipetting into an Eppendorf tube, placed on ice and then stored at −70° C. until assay.

Saliva Collection using a Modified Procedure

To collect a larger volume of saliva, the oral cavity of the horse was swabbed all around with a cotton bud. The saliva soaked cotton bud was then placed in a 20 ml syringe barrel into which the plunger is then applied to squeeze the saliva from the cotton bud into a sterile collection tube. Using this procedure, a total of 0.25-1.5 mL of horse saliva can be collected. After centrifugation to remove debris, the saliva is then stored at −20° C. until assay.

### Example 2

ELISA Assay for IgA and IgG

1. Wells of a 96-well microtitre plate were coated with rabbit anti-IgG or anti-IgA (Bethyl Laboratories, Sydney NSW) at 1:1000 dilution in sodium borate buffer (pH8) overnight at 4° C.

2. After washing with 1% Tween 20 in PBS, the wells were blocked with 2% BSA (100 μL) in PBS for 1 hr at room temperature.

3. After a removal of blocking buffer, the wells were washed in PBS/Tween and the excess buffer removed by tapping on a paper towel.

4. Horse saliva at 1:2000-1:5000 dilution in PBS/Tween was added in 100 μL aliquots to each well and incubated for 1 hr at room temperature. Horse serum (Hunter Antisera, Newcastle, NSW) was added as a calibrator for IgG and IgA.

5. After the wells were washed in PBS/Tween and peroxidase conjugated sheep anti-IgA or anti-IgG (ICN Biomedicals, Seven Hills, NSW) in 100 μL. aliquots were added to each well.

6. After incubation for 1 hr, the wells were washed in PBS/Tween followed by adding to each well 100 μL aliquots of an enzyme substrate tetramethybenzidine (TMB) solution.

7. After 20 mins incubation, the colour reaction was stopped by adding 2M H₂SO₄ solution and the absorbance read at 450 nm in an ELISA plate reader. The results were expressed as ELISA UNITS (EU) being absorbance of test samples interpolated from the linear portion of the calibration curve constructed from dilutions of a standard horse serum with a concentration of IgA at 1,000 EU.

The ELISA assay may be used in many different formats, eg. visual readout-type, dip-stick, and the like, all known to those skilled in the art. Other suitable immunoassays may also be used and again will be known to those skilled in the art.

### Example 3

Salivary Immunoglobulin Levels in Horses

The range of IgA and IgG concentrations in saliva from 28 ‘resting’ thoroughbred horses after exercise or polo competition is shown in Table 1. The range of IgA concentrations except for one horse were significantly higher than IgG (p<0.011), suggesting that the presence of local production and active transport of IgA into secretion.

TABLE 1
HORSE SALIVA IgA and IgG

| | IgA EU/mL | IgG EU/mL |
|---|---|---|
| | 46.0 | 3.80 |
| | 38.1 | 9.70 |
| | 340.0 | 15.60 |
| | 16.4 | 2.50 |
| | 542.0 | 7.60 |
| | 14.8 | 11.20 |
| | 118.5 | 6.80 |
| | 11.1 | 3.84 |
| | 43.2 | 6.91 |
| | 6.9 | 8.20 |
| | 43.4 | 3.20 |
| | 18.3 | 3.50 |
| | 170.0 | 14.00 |
| | 12.7 | 1.90 |
| | 5.1 | 1.23 |
| | 20.6 | 8.80 |
| | 43.0 | 8.10 |
| | 125.0 | 13.80 |
| | 22.7 | 4.60 |
| | 35.5 | 20.10 |
| | 579.0 | 57.50 |
| | 19.6 | 4.40 |
| | 7.8 | 4.60 |
| | 19.2 | 8.70 |
| | 23.9 | 2.90 |
| | 26.3 | 5.60 |
| | 22.0 | 2.70 |
| | 8.2 | 23.50 |
| Mean ± SEM | 84.97 ± 28.6 | 9.47 ± 2.1 |
| | (n = 28) | (n = 28) |

The lower levels of salivary IgA may reflect slow recovery from the effect of training or after polo competition on the day when the saliva samples were collected. Evidence from human studies showed that intense exercise or overtraining in elite athletes led to immunosuppression characterised by a slower recovery of salivary IgA levels and a susceptibility to infection. Similar effects due to stress may result in horses following training or competition.

### Example 4

Relationship between Salivary IgA Levels and Performance in Horses

Salivary IgA was collected from another group of horses in which assessment was also made of their performance using a visual analogue scale graded from 1 (lowest) to 5 (highest) performance as follows:

1=poor; 2=good; 3=very good; 4=excellent; 5=outstanding

Performance was judged in relation to that expected from a particular animal.

The data is presented in Table 2 below. Figures in the table represent salivary IgA levels in EU/ml. For purposes of statistical analysis of the data where appropriate, score groups 4 and 5 were combined and compared with group 1.

TABLE 2
Relationship between performance score and salivary IgA
levels

| | PERFORMANCE SCORE (1-5) | | | | |
|---|---|---|---|---|---|
| HORSE NAME | 1 | 2 | 3 | 4 | 5 |
| Santa Rigalia | | | | 132 | |
| Spirit of Fire | | | 59 | | |
| I'm on Fire | | | | | 801 |
| It is so | | 119 | | | |
| Constraint | | | | | 576 |
| Horse Shoe Bend | | | | | 885 |
| Fire Chief | | 141 | | | |
| Breaker Moran | | | | | |
| Bluey | | 147 | | | |
| Gran Tourismo | | | | | |
| Paint it Silver | 75 | | | | |
| Nacka-Yama | | | 434 | | |
| Copernicus | | | | 86 | |
| Straight Answer | 19 | | | | |
| Mary Alice | 8 | | | | |
| Summer Silence | 46 | | | | |
| Crystal Glow | | | | | |
| Jasha Boy | | | | | |
| Lucrative Medal | 11 | | | | |
| City Beach | 16 | | | | |

High score vs low score analysis (unpaired t-test for Elisa Units):

| Mean Diff | DF | t-Value | P-Value |
|---|---|---|---|
| 476 | 8 | 2.865 | 0.021 |

Group information for Elisa Units

| | Count | Mean | Variance | Std Dev | Std Err |
|---|---|---|---|---|---|
| High score | 5 | 496 | 137835.5 | 371.262 | 166.033 |
| Low score | 5 | 20 | 229.5 | 15.149 | 6.775 |

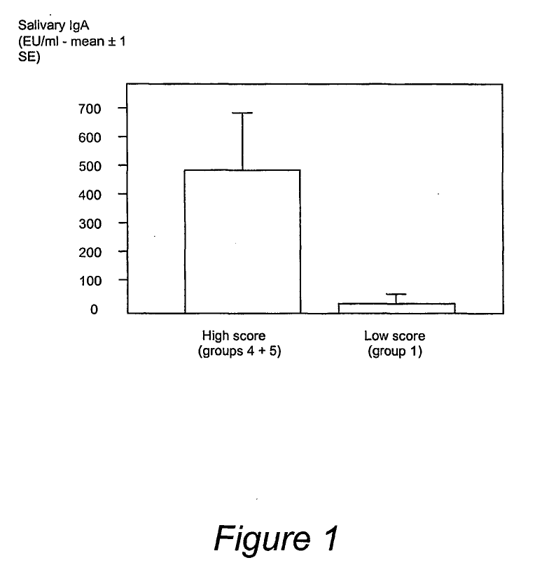

FIG. 1 shows the summarised data in graphical form.

This data suggests that a particular cut-off value of salivary IgA, for example 100 EU/ml in the present example 10, could be used to identify all or the majority of poor performers which may require special attention.

Further, high levels of salivary IgA, for example 400 EU/ml or greater, in good performers can indicate that optimal training programs can be implemented but that consideration can be given to increasing training. On the other hand high levels of salivary IgA in not so good performers can be indicative of the need to increase or optimise training programs. It would be particularly useful to make use progress figures, for example weekly or monthly assessment, to detect trends, either downwards, stable or upwards, which can be integrated with training programs.

Similarly, low levels of salivary IgA, for example less than 150 EU/ml, in good performers may indicate that the animal is at risk of loss of performance or has poor potential to perform and thus must be treated with care. Training may need to be modified or suspended and salivary IgA levels monitored closely. In poor performers low IgA levels can be indicative of the need for immediate attention and considerations of allowing the animal extensive rest periods. Levels of salivary IgA can be monitored as a guide to recovery.

Intermediate levels of salivary IgA may require closer monitoring to establish trends as discussed above. Whatever the performance level, IgA levels should be monitored to determine individual profiles.

### Example 5

IgA Levels and Performance in Race Horses Following Intense Training

Forty three thoroughbred race horses from two top racing stables in Sydney were assessed for performance and salivary IgA levels over a 8 week period with daily intense training at a race track. Saliva samples were collected weekly from individual horses before (week 1) and during training (week 2-8). The horses' performances were assessed by two trainers and recorded with no knowledge from the laboratory staff performing the IgA tests and vice versa. At the end of 8 weeks, all saliva samples were measured for equine IgA levels by the ELISA test described above. Saliva IgA levels with the mean performance indicators from 1 to 5 from two trainers were then matched for each horse.

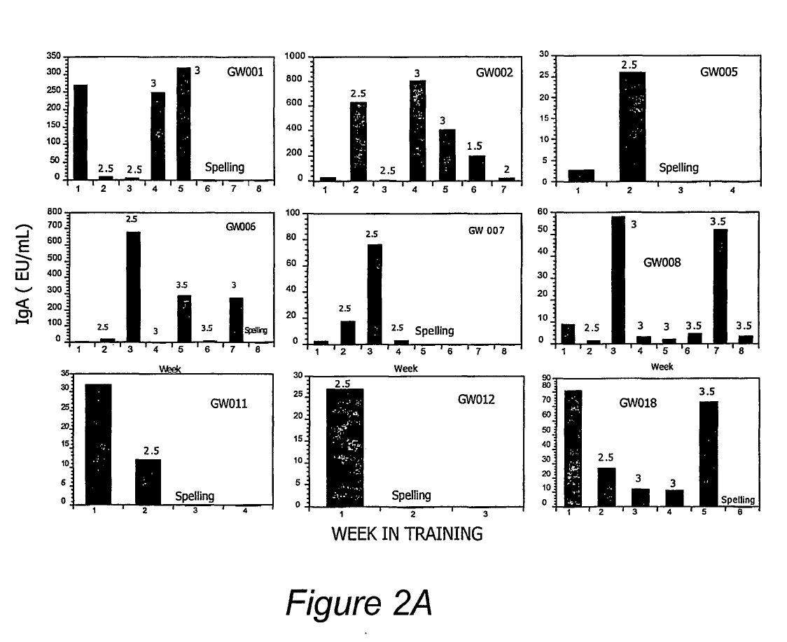

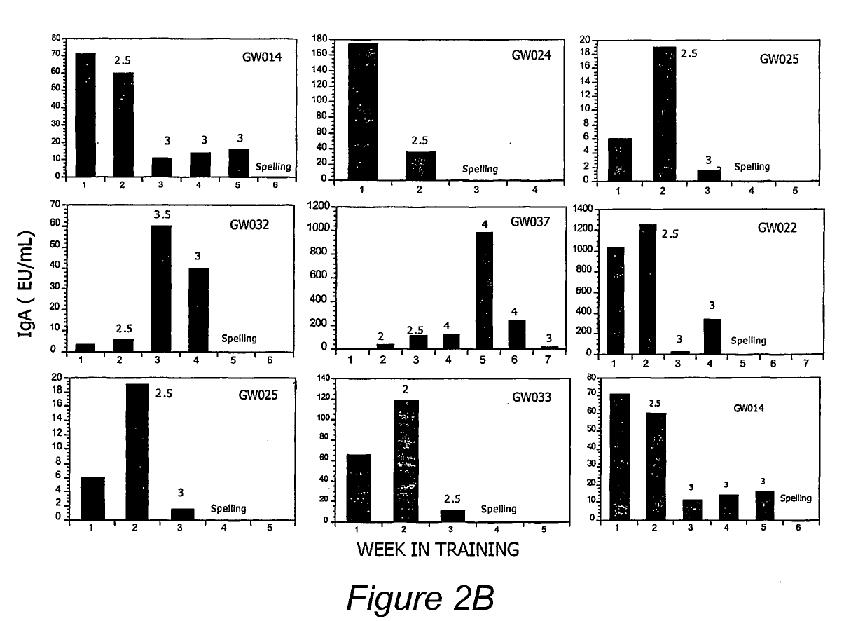

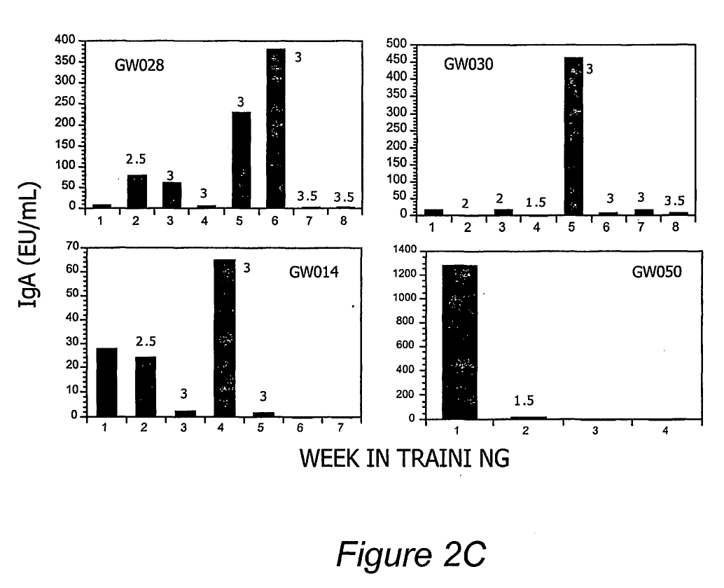

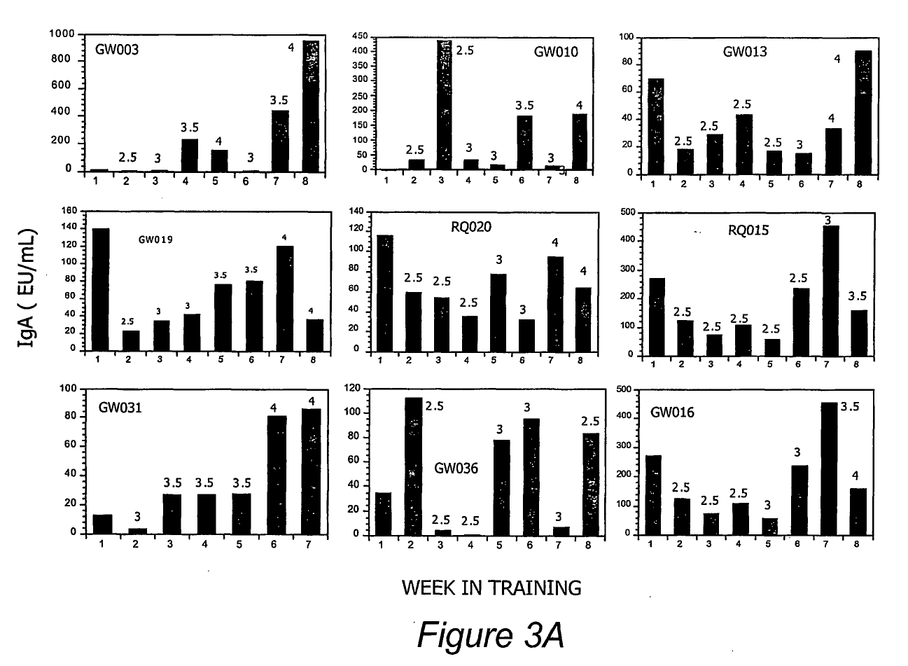

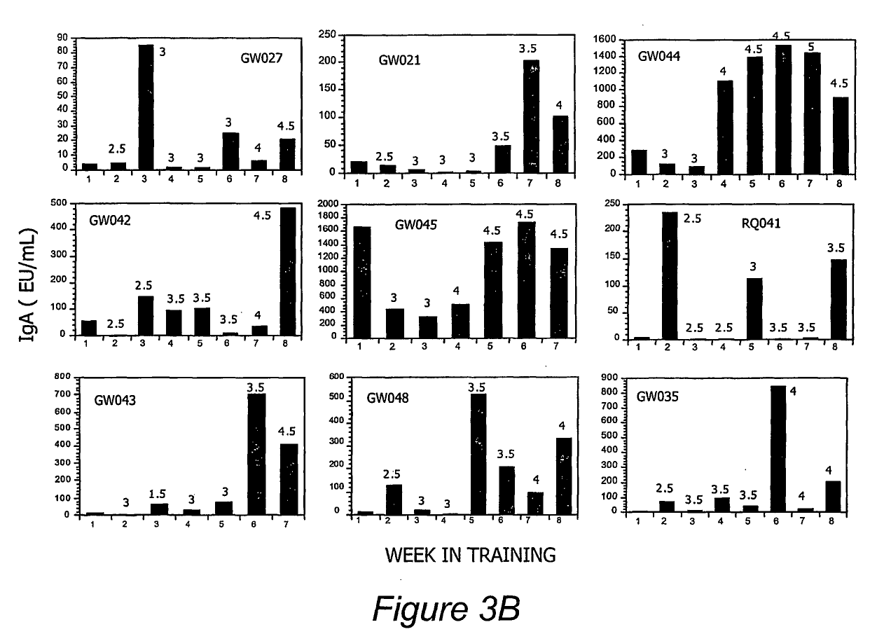

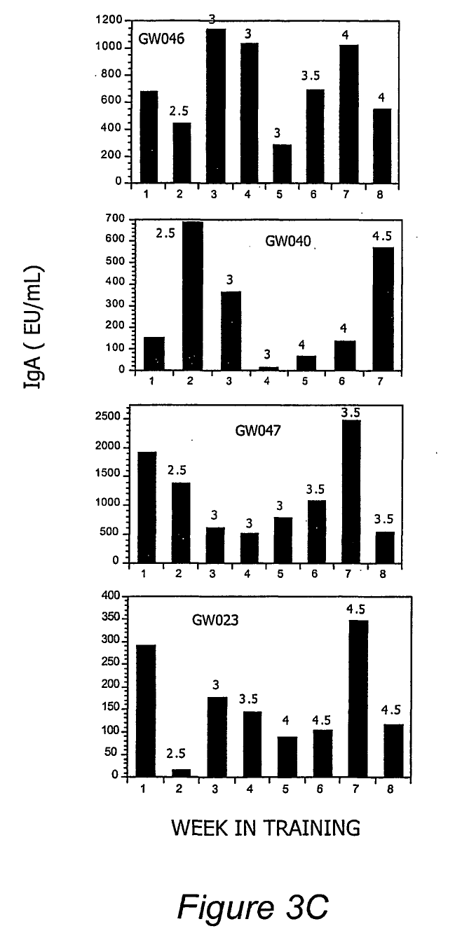

As shown in FIGS. 2A, 2B and 2C, 22 of 43 horses evaluated failed to complete the training program due to spelling (fatigue, inactivity or physical injury) with 13 horses (eg. horses GW001, GW005, GW011, GW012, GW024, GW025, GW032, GW 024) failed to progress beyond 4 weeks following the training program. In most cases, their IgA levels were lowest at the time when they were withdrawn from the training program compared with levels before training or during training. While there was a rise and fall in the IgA levels with training, horses (eg. GW002, GW006, GW018, GW014, GW037, GW014, GW028, GW030) which failed to perform over the 8 week training program also had poor recovery of salivary IgA levels compared with previous threshold levels. In marked contrast, 21 horses which completed the training program not only had higher levels of IgA (eg. horses GW010, GW003, RQ015, GW016, GW019, GW044, GW045, GW042, GW048, GW046) but also had a better recovery of salivary IgA levels from intense training and a superior performance as assessed by the trainers (FIGS. 3A, 3B and 3C). Taken together, the data provides the first evidence of a method for assessing the impact of a stressor such as intense training on a horse's performance or fatigue levels or potential to perform based on the determination of salivary IgA levels.

Further, the monitoring of drop in salivary levels of either IgA or IgG during training, followed by monitoring of recovery of salivary levels of these immunoglobulins over a specified period of time, can be used as a useful predictor of whether or not a horse will complete the training program and how a horse needs to be managed during training to achieve optimal performance. Also, starting levels of IgA are good indicators of sustained performance and/or suitability for a particular training program.

Preliminary analysis provides examples of how the data obtained in the study can be used. The IgA data indicate for example that:

It can be predicted whether or not a horse will successfully survive the training programme (note high VA scores around weeks 7 & 8)—ie 21/22 horses have VAS's of ≧3.5. The one horse with a VAS of <3.5 had a zero time low (<50 eu/ml) IgA level. VAS's in the first half of the training programme do not predict outcome.

Initial (pre-training) IgA levels correlated with outcome:

| | | | IgA | |
|---|---|---|---|---|
| | | | <50 EU | <25 EU |
| | | training | 12 | 10 |
| | | non completed | | |
| | | (21) (TNC) | | |
| (i) | Low levels | training | 9 | 7 |
| | | completed | | |
| | | (22) (TC) | | |
| | | | | >100 EU |
| | | | >100 EU | (+ >50 at week 2) |
| | | training | 3 | 0 |
| | | non completed | | |
| | | (21) (TNC) | | |
| (ii) | High levels | training | 9 | 7* |
| | | completed | | |
| | | (22) (TC) | | |

*The two horses with <50 EU at week 2, subsequently had rebound and then sustained IgA levels.

IgA profiling can also be useful, eg. none of those failing to complete training successfully, but who remained training for most of the time, had sustained levels (especially greater than 50 eu's) of IgA (seven subjects). Of those successfully completing training, however, 18 of 22 had sustained high levels of salivary IgA. There was threefold increase in frequency of consecutive weekly recoveries of IgA of <50 EUs in horses that eventually failed to complete training.

### Example 6

An Example of how IgA can be used for Monitoring Training

The absolute values of IgA for the purposes of this example are not critical and the cut off values are only a guide.

(i) Training onset and after one week's training (IgA levels given in EU's)

>100 EU with resting level at one week >50EU—very high chance of completing standard training (say over eight weeks) without modification.

(ii) Consecutive weekly levels of <50EU's identifies high risk of impaired performance.

Failure to sustain levels (especially >50EU's) particularly in second half of training predicts impaired performance.

(iii) Individual low levels (<50EU's). eg. for values<50EU's

VAS

| | <3.0 | >3.0 |
|---|---|---|
| Completed training | 12%* | 8% |
| Fail to complete training | 28% | 9% |

*% of total measurements

Twice the chance of ranking <3.0 on VAS at time of sample collection, but this is threefold if horse is in ‘fail to complete’ group.

(iv) Individual high levels (>100EU's)

Overall, threefold chance of a VAS of >3.0 (in ‘completed group’, this is fourfold). Thus—individual measurements have value in assessment of risk.

Trainer's assessment at particular points are not good predictors for completion of training programme (particularly in first half of programme).

The data obtained in this study includes other significant findings, which will be clearly recognised by those skilled in the art, and conventional statistical methods (with or without computer assistance) can be used for its analysis.

Thus, analysis of regular saliva IgA levels in the post training recovery phase during intense training has value in monitoring and predicting training intensity of race horses. Erratic, declining, and non-sustained levels (ie. falling below for example 50EU) best predict horses likely not to complete training.

Horses entering an intense training phase can be assessed as prone to impaired performance based on resting IgA levels and their potential performance assessed—a valuable indicator is a high resting IgA level (for example >100EU), particularly if good recovery levels after one week of intense training (for example ≧50EU). This represents high chance of quality outcome from training programme and represents 15-20% of all horses, but approximately 50% of total group completing training. It will be recognised that these figures relate to the particular example studied, and that cohorts of horses will vary depending on training patterns. It will also be recognised that horses found to be at risk of impaired performance because of baseline or profile patterns, may benefit from altered intensity of training.

Thus, measurement of salivary IgA and the establishment of trends and individual profiles can be used individually or in combination to determine and optimise management strategies with respect to training schedules, rest periods, psychological parameters, performance and the like.

Although the present invention has been described with reference to certain examples and preferred embodiments it will be understood that variations and modifications which are in keeping with principles and the spirit of the invention are also contemplated and will be understood by those skilled in the art.

The claims defining the invention are as follows:

1. Method for predicting performance potential, impaired performance, fatigue and/or potential susceptibility to infection in horses exposed to stressor(s) including:
(a) determination of immunoglobulin A (IgA) and/or immunoglobulin G (IgG) level in a training phase; and
(b) prediction of impaired performance, fatigue and/or susceptibility to infection by comparison of the training phase IgA and/or IgG level with a predetermined threshold value.

2. Method for assessing and/or monitoring suitability of a physical training program for horses, including:
(a) determination of IgA and/or IgG level in a pre-training phase;
(b) determination of IgA and/or IgG level at intervals during a training phase and
(c) comparing IgA level during training phase with IgA and/or IgG levels in pre-training phase and thereby assessing suitability of the physical training program.

3. Method for assessing the impact of stressor(s) on a horse's performance and/or fatigue levels, as defined herein, including:
(a) determination of salivary IgA and/or IgG level; and
(b) assessing the impact of the stressor or stressors on the horse's performance and/or fatigue levels by comparison of said IgA and/or IgG level with a predetermined threshold value.

4. Method for assessing potential susceptibility to respiratory infection in a horse, including:
(a) determining the salivary IgA and/or IgG level in early training phase; and
(b) predicting the potential susceptibility to respiratory infection by comparison of the early training phase IgA and/or IgG level with a predetermined threshold value.

5. Method for assessing potential susceptibility to fatigue/infection in horses, including:
(a) determining the IgA and/or IgG level after exposure to stressor(s);
(b) allowing a recovery period as herein defined;
(c) determining the IgA and/or IgG level after the recovery period;
(d) predicting the potential susceptibility to fatigue/infection by comparison of the IgA and/or IgG level at step (a) above with the IgA and/or IgG level at step (c) above.

6. Method for designing a training program for a horse, including the determination of salivary IgA and/or IgG levels at the time periods selected from one or more of the following:
(a) before training begins,
(b) in early training phase,
(c) during optimal training phase and/or during competition or severe physical stress.

7. Method for designing a training program for a horse, including:
(a) determining the salivary IgA and/or IgG level before training begins,
(b) determining the salivary IgA and/or IgG level in early training phase,
(c) determining the salivary IgA and/or IgG level during optimal training phase and/or during competition or severe physical stress,
(d) establish an IgA and/or IgG profile for the horse using levels obtained in steps (a) to (c) to design a training program which maintains stable levels of IgA and/or IgG.

8. A method according to claim 7, wherein the training program of step (d) is designed such that the level of IgA and/or IgG does not fall below a predetermined level.

9. Method for assessing performance potential of a horse including:
(a) determination of immunoglobulin A (IgA) and/or immunoglobulin G (IgG) level before training begins; and
(b) prediction of poor performance potential by comparison of the IgA and/or IgG level with a predetermined threshold value.

10. Method for assessing performance potential of a horse, including:
(a) determining the IgA and/or IgG level before training begins,
(b) determining the IgA and/or IgG level in early training phase,
(c) establishing an IgA and/or IgG profile for the horse using levels obtained in steps (a) to (b) and using said profile to predict poor performance potential by comparison of the IgA and/or IgG level with a predetermined threshold value.

11. Method for predicting the capacity of a horse to complete a training program, including:
(a) determining the IgA and/or IgG level before training begins,
(b) determining the IgA and/or IgG level in early training phase,
(c) establishing an IgA and/or IgG profile for the horse using levels obtained in steps (a) to (b) and using said profile to predict the capacity of a horse to complete a training program by comparison of the IgA and/or IgG level with a predetermined threshold value.

12. A method according to any one of claims 1 to 11 wherein the immunoglobulin level is determined in saliva.

13. A method according to any one of claims 1 to 12 wherein the immunoglobulin is IgA or a subclass thereof.

14. A method according to any one of claims 1 to 13 wherein the threshold value is a normal threshold value determined in samples obtained from normal horses.

15. A method according to any one of claims 1 to 13 wherein the threshold value is an internal individual threshold value.

16 A method according to any one of claims 1 to 15 wherein the saliva sample is whole unstimulated saliva.

17. A method according to any one of claims 1 to 16 wherein the horse is a racing or other competition horse.

18. A method according to any one of claims 1 to 17 wherein the stressor is known to influence the efficacy of the immune system.

19. A method according to any one of claims 1 to 18 wherein the immunoglobulin level is determined by an immunoassay selected from the group consisting of ELISA, RIA, immunodiffusion and the like.

20. A method according to any one of claims 1 to 19, wherein the saliva is analysed for the immunoglobulin content in situ by contacting an assay device or system with the saliva of a horse.

21. An assay device when used in a method according to any one of claims 1 to 20.
